(19) United States
(12) Patent Application Publication
(10) Pub. No.: US 2025/0205071 A1
(43) Pub. Date: Jun. 26, 2025

(54) COUNTERFORCE BRACE

(71) Applicant: Gordon Consulting, Inc., Kansas City, MO (US)

(72) Inventor: Howard A. Gordon, Kansas City, MO (US)

(21) Appl. No.: 19/073,799

(22) Filed: Mar. 7, 2025

Related U.S. Application Data
(63) Continuation-in-part of application No. 16/789,048, filed on Feb. 12, 2020.

(51) Int. Cl. A61F 5/01

(52) U.S. Cl. CPC A61F 5/0111

(57) ABSTRACT

Provided herein are a foot and ankle bracing device and methods of using the device to anatomically stabilize the three primary aspects of foot movement desired to protect the ankle complex during load bearing forces.

### CROSS-REFERENCE TO RELATED APPLICATIONS

The present application is a continuation-in-part of U.S. application Ser. No. 16/789,048 filed on Feb. 12, 2020, the entire contents of which are hereby incorporated by reference.

### FIELD OF THE INVENTION

The present invention relates generally to a stabilizing foot and ankle bracing device.

### BACKGROUND OF THE INVENTION

The supination of a foot combines inward rotation at the ankle, adduction of the hindfoot, inversion of the forefoot, and medial arch elevation. In short, supination produces a medial (inward) movement at the talocalcaneal (sub talus) joint known as inversion as well as similar movement between the hindfoot (talus) and midfoot (navicular) bones.

A foot that supinates appears to naturally favor the lateral region or outside edge of the foot. A certain amount of supination is normal during the toe-off stage of running, i.e., when the heel first lifts off the ground until the end of the step. During excessive supination, the lateral structures of the foot tighten which may rupture or avulse portions of the lateral collateral ligaments resulting in a “rolling over” of the ankle or ankle sprain.

The pronation of a foot produces opposite foot behavior of the subtalar joint, including adduction of the forefoot, eversion of the hindfoot and dorsiflexion. Pronation produces a lateral (outward) movement of the foot at the subtalar joint known as eversion as well as similar hindfoot and midfoot movements. A foot that pronates appears to favor the inside or medial edge of the foot naturally. The foot naturally pronates to absorb the shock resulting from the heel hitting the ground. Excessive pronation increases the stress on the inside/medial portion of the foot which may lead to various injuries such as arch strain, heel pain, inner ankle pain and shin splints.

Accordingly, it is desirable to preclude excessive pronation and/or supination of the ankle joint and foot. Thus, various devices have been proposed to prevent ankle and foot injuries by attempting to maintain the foot in a “neutral position.” Although assumedly effective in their operation, such devices have failed to simultaneously provide effective stabilization to the forefoot, transverse arch, and ankle complex for the safety of the foot from heel-strike to toe-off thereby providing proper anatomical foot movement during walking, running or jumping movements.

There is a need for a device that provides proper anatomical foot movement during walking, running, or jumping movements by simultaneously stabilizing the forefoot, transverse arch, and ankle complex for safety of the foot from heel-strike to toe-off.

### SUMMARY

Provided herein is a foot and ankle stabilizing brace. The foot and ankle stabilizing brace can include a body, a medial countervailing force strap, a lateral countervailing force strap, a first bi-lateral heel strap, and a second bi-lateral heel strap. The body can include a first aperture operable to receive a foot of a wearer and a second aperture operable to receive a foot of a wearer. The first aperture can be above an ankle of the wearer in use. The second aperture can be operable to encompass distal ends of metatarsals of the wearer in use. The medial countervailing force strap can be operable to couple to the body near a medial side of the first aperture and near a medial side of the second aperture. The lateral countervailing force strap can be operable to couple to the body near a lateral side of the first aperture and near a lateral side of the second aperture. The first bi-lateral heel strap can be operable to couple to the body near a lateral side of a heel of the wearer and at a medial side of the body above the ankle of the wearer. The second bi-lateral heel strap can be operable to couple to the body near a medial side of the heel and at a lateral side of the body above the ankle. The first bi-lateral heel strap can be operable to wrap around a hindfoot and the ankle of the wearer. The second bi-lateral heel strap can be operable to wrap around the hindfoot and the ankle.

In some aspects, the first bi-lateral heel strap and the second bi-lateral heel strap can be operable to secure a subtalar joint of the wearer in subtalar neutral, thereby protecting posterior and anterior tabula fibular and calcaneus fibular ligaments associated with ankle injuries. In some aspects, the medial countervailing force strap can be operable to wrap around the foot of the wearer. In some aspects, the medial countervailing force strap extends from the medial side of the second aperture, over the foot to a lateral side of the body, and under the foot to the medial side of the first aperture. In some aspects, the medial countervailing force strap includes a fastener operable to couple to a corresponding fastener of the body at the lateral side of the body. In some aspects, the lateral countervailing force strap is operable to wrap around the foot of the wearer. In some aspects, the lateral countervailing force strap extends from the lateral side near the second aperture, over the foot to a medial side of the body, and under the foot to the lateral side near the first aperture. In some aspects, the lateral countervailing force strap includes a fastener operable to couple to a corresponding fastener of the body at the medial side of the body.

In some aspects, the foot and ankle stabilizing brace further includes a tongue and a set of laces attached to the body. The set of laces can be operable to secure the body and the tongue around the foot of the wearer.

In some aspects, the medial countervailing force strap and the lateral countervailing force strap can be operable to provide countervailing forces along the foot and the ankle to resist excessive supination and pronation thereof. In some aspects, the first bi-lateral heel strap and the second bi-lateral heel strap can provide additional control of inversion/eversion movement of a forefoot of the wearer. In some aspects, the additional control provided by the first bi-lateral heel strap and the second bi-lateral heel strap can prevent excess inversion/eversion, excess pronation/supination, and excess dorsiflexion/plantarflexion. In some aspects, the first bi-lateral heel strap can cross the second bi-lateral heel strap near the midfoot.

Further provided herein is a method for preventing excessive supination and/or pronation of an ankle. The method can include providing the foot and ankle stabilizing brace, extending the foot through the first aperture, extending the foot through the second aperture, wrapping the lateral countervailing force strap around the foot, wrapping the medial countervailing force strap around the foot, wrapping the first bi-lateral heel strap around the wearer, and wrapping the second bi-lateral heel strap around the wearer.

Wrapping the medial countervailing force strap around the foot can include extending the medial countervailing force strap from the medial side of the second aperture to a lateral side of the body, underneath an arch of the wearer to the medial side of the body, and up the medial side of the body near the first aperture. Wrapping the lateral countervailing force strap around the foot can include extending the lateral countervailing force strap from the lateral side near the second aperture to a medial side of the body, underneath the arch to the lateral side of the body, and up the lateral side of the body near the first aperture. Wrapping the first bi-lateral heel strap around the wearer can include extending the first bi-lateral heel strap from the lateral side of the heel, around the ankle, around the midfoot, and around the ankle to the medial side of the body above the ankle. Wrapping the second bi-lateral heel strap around the wearer can include extending the second bi-lateral heel strap from the medial side of the heel, around the ankle, around the midfoot, and around the ankle to the lateral side of the body above the ankle.

In some aspects, the first bi-lateral heel strap and the second bi-lateral heel strap can secure a subtalar joint of the wearer in subtalar neutral, thereby protecting posterior and anterior tubular fibular and calcaneus fibular ligaments associated with ankle injuries. In some aspects, the medial countervailing force strap and the lateral countervailing force strap can be operable to provide countervailing forces along the foot and the ankle to resist excessive supination and pronation thereof.

In some aspects, the first bi-lateral heel strap and the second bi-lateral heel strap can provide additional control of inversion/eversion movement of a forefoot of the wearer. In some aspects, the additional control provided by the first bi-lateral heel strap and the second bi-lateral heel strap prevents excess inversion/eversion, excess pronation/supination, and excess dorsiflexion/plantarflexion. In some aspects, the first bi-lateral heel strap crosses the second bi-lateral heel strap near the midfoot. In some aspects, the foot and ankle stabilizing brace further includes a tongue and a set of laces attached to the body. The set of laces can be operable to secure the body and the tongue around the

### BRIEF DESCRIPTION OF THE DRAWINGS

FIG. 1 is a diagrammatic view showing pronation of the right foot from the rear.

FIG. 2 is a diagrammatic view showing supination of the right foot from the rear.

FIG. 3 is a top/dorsal view of the right foot of the wearer. Metatarsals and phalanges of the foot of the wearer and the second edge of the body are shown in phantom lines.

FIG. 4 is a perspective view of the medial side of the right foot of the wearer in the brace. The medial and metatarsal straps and the circumferential strap, partially shown behind the brace, are disengaged.

FIG. 5 is a perspective view of the medial side of the right foot of the wearer in the brace. The circumferential strap is engaged around the brace.

FIG. 6 is a perspective view of the medial side of the right foot of the wearer in the brace. The circumferential strap is engaged around the brace. The medial strap is extended across the dorsal aspect of the forefoot, rearwardly underneath the arch of the wearer towards the lateral side of the heel, and upwardly along the medial side of the ankle joint region.

FIG. 7 is a perspective view of the lateral side of the right foot of the wearer in the brace. The circumferential strap is engaged around the brace. The medial strap is extended across the dorsal aspect of the forefoot.

FIG. 8 is a perspective front view of the right foot of the wearer in the brace. The circumferential strap is engaged around the brace. The medial and lateral straps are wrapped and secured around the foot.

FIG. 9 is a bottom view of the right foot of the wearer in the brace. The medial strap is shown extending from the second edge of the brace underneath the arch of the wearer towards the lateral side of the heel. The lateral strap is shown extending from the second edge of the brace underneath the arch of the wearer towards the medial side of the heel.

FIG. 10 is a lateral view of the foot of the wearer secured in the brace.

FIG. 11 is a bottom view of the right foot of the wearer in the brace showing the lateral and medial straps attached to the second edge.

FIG. 12 is a dorsal view of the right foot of the wearer in the brace. The medial and lateral straps are disengaged. Arrows depict the direction of extension of the lateral and medial straps around the foot.

FIG. 13 is a side view of the lateral side of the right foot of the wearer in the brace. The circumferential strap is engaged around the brace. The medial and lateral straps are wrapped around the foot, drawn upwards and attached to the complementary means of attachment on the sides of the brace above the ankle joint.

FIG. 14 is a bottom view of the right foot of the wearer in the brace. The medial and lateral straps are disengaged and indicate the means for attaching the straps to the brace above the ankle joint.

FIG. 15 is a rear view of the right foot of the wearer with the brace secured thereon.

FIG. 16 is a side view of the lateral side of the right foot of the wearer in the brace. The circumferential strap is positioned to engage around the brace. The medial is disengaged.

FIG. 17 is a view of the pattern of the circumferential strap.

FIG. 18 is a view of the pattern of the first integral sleeve.

FIG. 19 is a view of the pattern of the second integral sleeve.

FIG. 20 is a view of the pattern of the foot plate.

FIG. 21 is a view of the pattern of the lateral and medial countervailing force straps.

FIG. 22 is a view of the pattern of the lateral and medial heel lock and torque strap.

FIG. 23 is a lateral side view of an aspect of the brace showing only the first and second bi-lateral heel straps. The second end of the first bi-lateral heel strap is shown unattached and the second end of the second bi-lateral heel strap is shown attached to the body of the brace at a location above the ankle joint complex at the lateral side of the body of the brace.

FIG. 24 is a lateral side view of an aspect of the brace comprising countervailing force straps and bi-lateral heel straps.

FIG. 25 illustrates the brace in one example.

FIG. 26 illustrates the brace in one example.

FIG. 27 illustrates the brace in one example.

FIG. 28 illustrates the brace in one example.

FIG. 29 illustrates the brace in one example.

FIG. 30 illustrates the brace in one example.

FIG. 31 illustrates the brace in one example.

FIG. 32 illustrates the brace in one example.

### DETAILED DESCRIPTION

The devices and methods described herein will be understood from the accompanying drawings, taken in conjunction with the accompanying description. It is noted that, for purposes of illustrative clarity, certain elements in various drawings may not be drawn to scale. Several variations of the system are presented herein. It should be understood that various components, parts, and features of the different variations may be combined together and/or interchanged with one another, all of which are within the scope of the present application, even though not all variations and particular variations are shown in the drawings. It should also be understood that the mixing and matching of features, elements, and/or functions between various variations is expressly contemplated herein so that one of ordinary skill in the art would appreciate from this disclosure that the features, elements, and/or functions of one variation may be incorporated into another variation as appropriate, unless described otherwise.

The present disclosure concerns a foot and ankle stabilizing brace incorporating strapping mechanisms that, when used together, can anatomically stabilize the three primary aspects of foot movement desired to protect the ankle complex during load-bearing forces, thereby reducing the probability and/or potential and degree of injury. More specifically, the inventor surprisingly discovered that the brace provides significantly improved protection for the forefoot when compared to foot braces available at the time the invention was made. The significantly improved protection provided by the claimed foot brace is due to the ability of the foot brace to provide improved stabilization of the three primary aspects of foot movement. More specifically, the inventor discovered that the brace of the instant disclosure can prevent excess inversion/eversion, excess pronation/supination, and excess dorsiflexion/plantarflexion. By providing the improved protection, the brace of the instant disclosure can significantly improve prevention of common foot injuries that include “rolling over” of the ankle or ankle sprain, arch strain, heel pain, inner ankle pain, and shin splints. The brace and methods of using the brace are described below.

### I. Brace

One aspect of the instant disclosure encompasses a foot and ankle stabilizing brace. The foot and ankle stabilizing brace comprises a body operable to be worn on the foot of a subject and strapping mechanisms engineered to anatomically stabilize the three primary aspects of foot movement desired to protect the ankle complex during load-bearing forces. The strapping mechanisms comprise a combination of countervailing force straps with heel lock and torque straps or with bi-lateral heel straps.

### (a) The Body

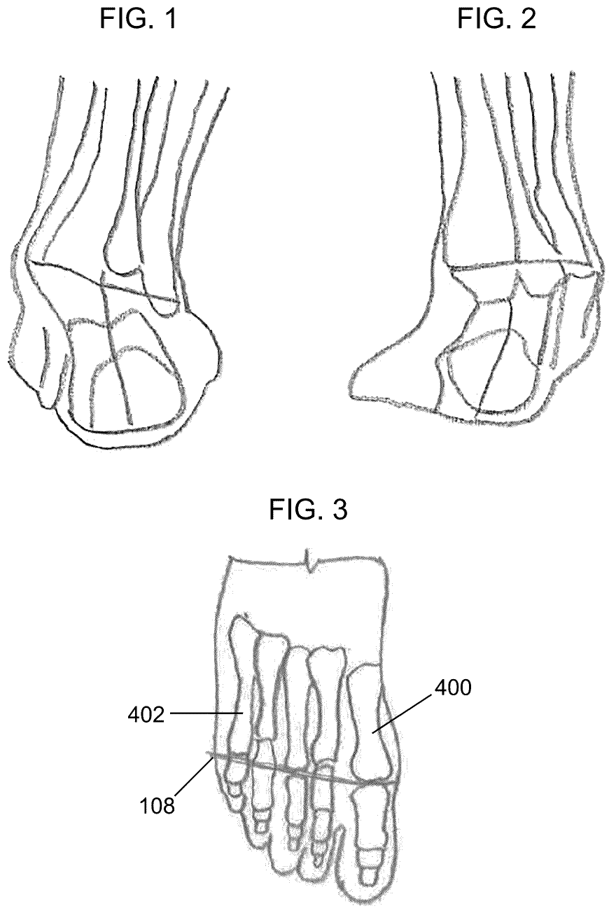

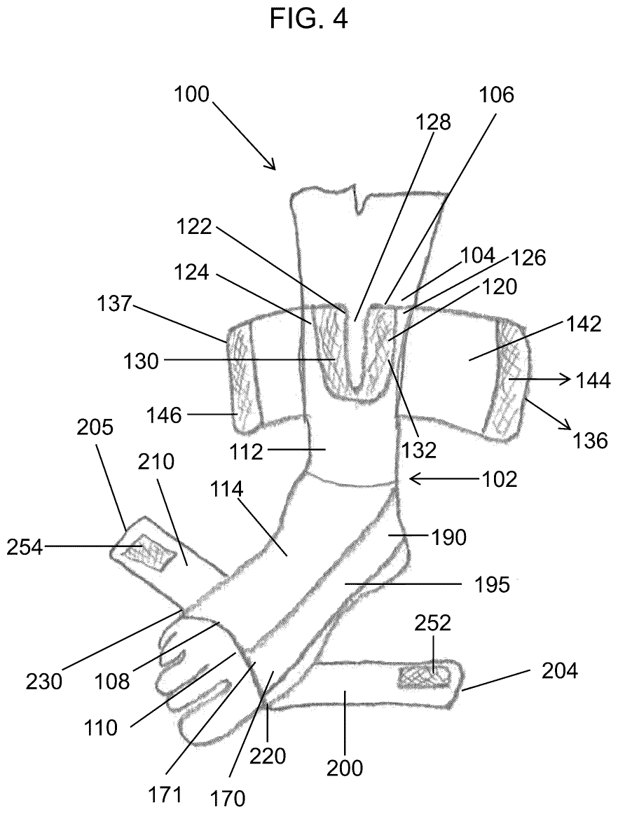

Turning more particularly to the drawings, brace 100 comprises a sock-like body 102. The body 102 provides a compression fit about the ankle joint complex and forefoot of the wearer. The body 102 provides a compression fit about the ankle joint complex of the wearer and extending to a compression fit surrounding the forefoot of the wearer. In some aspects, the body 102 comprises a first integral sleeve 112 providing a compression fit at and above the ankle joint complex of the wearer and a second integral sleeve 114 providing a compression fit on the forefoot of the wearer. The forefoot is described as that region of the foot extending from the distal/head ends of the first 400 and fifth metatarsals 402 through the phalanges (FIGS. 1-3). The body 102 comprises a first free edge 104 circumscribing a first aperture operable to encompass the foot at a superior location above the ankle joint complex. The body 102 further comprises a second free edge 108 circumscribing a second aperture operable to encompass the foot adjacent the distal ends/heads of the first 400 and fifth 402 metatarsals. The first free edge 104 and the second free edge 108 can be operable to receive the foot of the wearer. The wearer's foot can be inserted through the first free edge 104 through the body 102 and through the second free edge 108. When the brace 100 is installed on the wearer (e.g., in use), the first free edge 104 (e.g., first aperture) can be located above the ankle of the wearer. When the brace 100 is installed on the wearer (e.g., in use), the second free edge 108 (e.g., second aperture) can be operable to encompass distal ends of the metatarsals of the wearer. The first free edge 104 can be on an opposite end of the body 102 from the second free edge 108, as illustrated, for example, in FIG. 4.

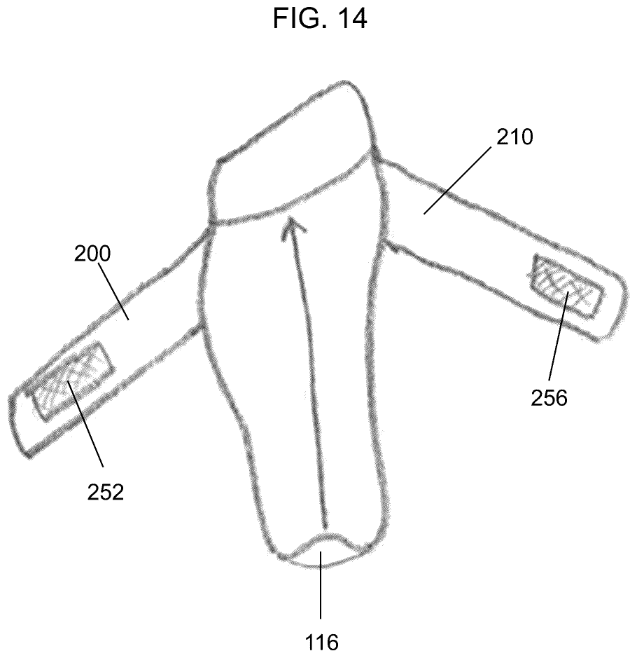

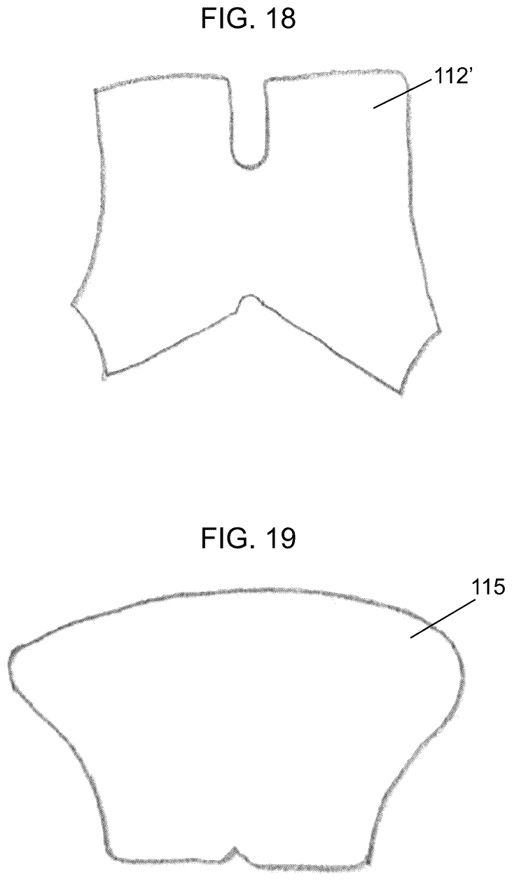

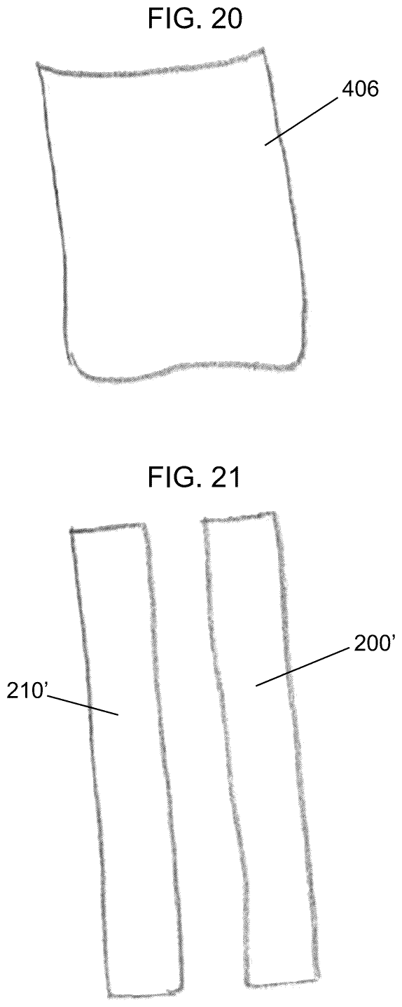

In some aspects, the second free edge 108 does not extend beyond the distal ends/heads of the first 400 and fifth 402 metatarsals as it may reduce dorsal flexion in the foot. As such, upon slipping the body 102 over the foot, the body 102 presents a compression fit about the ankle joint complex and forefoot of the wearer between the first free edge 104 and the second free edge 108. In some aspects, body 102 can further comprise an aperture 116 at the heel to allow for extension of the heel therethrough (shown in FIGS. 8, 14, and 18). When the body 102 comprises a first integral sleeve 112 connected to a second integral sleeve 114, the first integral sleeve 112 can be constructed from pattern 112′ (FIG. 18), and the second integral sleeve 114 can be constructed from dorsal pattern 115 (FIG. 19) and foot plate pattern 406 (FIG. 20).

The initial extension of the foot through the first free edge 104 (e.g., first aperture) can be enhanced by a closure system 120 (FIG. 4). The closure system 120 comprises a notch 122, which extends from the first free edge 104 of first integral sleeve 112 and partially bisects the first integral sleeve 112 between flaps 124 and 126. In some aspects, the notch 122 is U-shaped, although other shapes can also be appropriate and will be recognized by individuals of skill in the art. As such, the closure system 120 allows the user to increase the initial opening presented by the upper first free edge 104 such that feet of various sizes may be initially extended through this first free edge 104 for ultimate extension of the phalanges 404 through the downstream of the second free edge 108. The closure system 120 comprises means for fastening flap 124 to flap 126 to secure the first free edge 104 around the foot of the wearer above the ankle joint complex. The closure system 120 enables the user to adjust the compression of the first integral sleeve 112 about the ankle joint region as the degree of overlap of flaps 124 and 126 can be selectively adjusted. In other aspects, the extension of the foot through the free edge 104 (e.g., first aperture) can be enhanced using the set of laces described further herein.

In some aspects shown in the figures herein, the means for fastening flap 124 to flap 126 is a loop and hook system, such as Velcro™ fastening material. However, one of ordinary skill in the art will appreciate that other means of fastening may be used, including, but not limited to, buttons, snaps, zippers, buckles, semi-permanent adhesives, laces, or any combination of the above. In some aspects shown in the figures herein, the means for fastening flap 124 to flap 126 is one or more laces. The one or more laces can be interlaced with the fastening flap 124 and flap 126. The one or more laces can be operable to secure the body around the wearer above the ankle.

As shown in the aspect depicted in the figures, the fastening system can include tab 130 which is secured along the longitudinal extent of the notch 122 on flap 124, and tab 132 which is secured along the longitudinal extent of the notch 122 on flap 126. Tabs 130 and 132 have complementary hook and loop fastener material attached thereon for securing flap 124 to flap 126.

In some aspects, a tongue 128 is attached to the interior of first integral sleeve 112 for extension along notch 122 so as to preclude the flaps 124 and 126, and tabs 130 and 132 of the closure system 120 from directly contacting the user. The tongue 128 can be attached to the body 102. The tongue 128 can be operable to span an area between the notch 122 and the wearer. The tongue 128 can prevent unwanted contact between the closure system 120 and the wearer's skin.

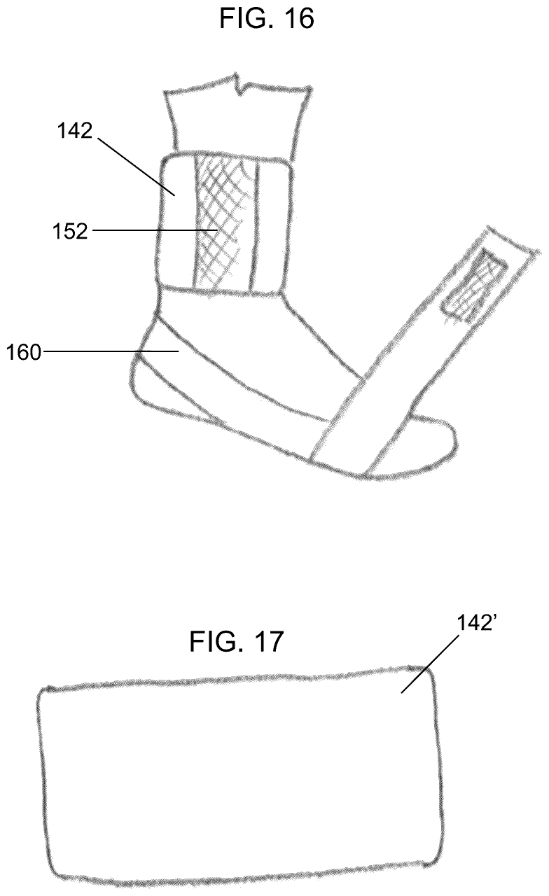

As illustrated in FIGS. 4 and 16, the brace can further include a circumferential strap 142 for wrapping around the first integral sleeve 112 about the ankle joint complex of the foot of the wearer. The circumferential strap 142 is made from pattern 142′ and is generally rectangular in shape, although other appropriate shapes can be envisioned, as illustrated in FIG. 17. For instance, the strap can have a shape configured to follow the contours of a foot about the ankle complex. The circumferential strap 142 comprises a left edge 136, and a right edge 137. The circumferential strap 142 comprises a closure system for securing the strap around the first integral sleeve 112 above the ankle joint complex of the foot of the wearer adjacent the first free edge 104. The closure system comprises means for fastening (e.g., coupling) the left edge 136 to the right edge 137 to secure the circumferential strap 142 around the first integral sleeve 112 and the foot. The fastening of the left edge 136 to the right edge 137 enables the user to adjust the compression of the circumferential strap 142 about the ankle joint region as the degree of overlap of the left edge 136 and the right edge 137 can be selectively adjusted. The degree of overlap of the left edge 136 to the right edge 137 adjusts the compressive fit of circumferential strap 142, about the ankle joint and thus maintains the circumferential integrity of the brace about the ankle joint complex. In some aspects, shown in the figures herein, the means for fastening the left edge 136 and the right edge 137 is a loop and hook system, such as Velcro™ fastening material. However, one of ordinary skill in the art will appreciate that other means of fastening may be used, including, but not limited to, buttons, snaps, zippers, buckles, semi-permanent adhesives, laces, or any combination of the above. As shown in the aspect depicted in the figures, the fastening system can comprise a first tab 144 which is secured along the length of the left edge 136 of circumferential strap 142, and a second tab 146 which is secured along the length of the right edge 137 of circumferential strap 142. First tab 144 and second tab 146 have complementary hook and loop fastener material attached thereon for securing the left edge 136 and the right edge 137. Accordingly, the circumferential strap 142 is wrapped circumferentially about the ankle joint complex and the left edge 136 and the right edge 137 are attached one to the other. The circumferential strap 142 can be made from pattern 142′. In some aspects, the circumferential strap 142 is permanently attached at the rear of the first integral sleeve 112 adjacent first free edge 104. Alternatively, the circumferential strap 142 can be removably attached to the back of the first integral sleeve 112 using other means for attaching, including but not limited to, buttons, snaps, buckles, zippers, semi-permanent adhesives, or combinations of the above.

Any material capable of providing a sufficiently compressive fit, strength, and comfort can be appropriate for constructing the sock like body 102 of the brace. In some aspects, the body 102 is constructed of PES Oxford fabric with PVC coating. Any material of sufficient strength can be appropriate for constructing the straps. In some aspects, straps are constructed of multiple strength polyester materials. In some aspects, the brace is constructed using sewn seams for structural strength.

### (b) Countervailing Force Straps

In some aspects, the brace comprises countervailing force straps. The straps are anatomically lengthened, positioned and attached to the body of the device at the respective distal ends of the first and fifth metatarsals to affect the following ankle complex securing results. Initially, each strap will stabilize the forefoot from excess inversion/eversion movement, thereby commence securing the forefoot from unwarranted rolling. Next, said straps crisscross over the dorsal aspect of the foot, securely, flowing under the transverse arch to reduce excess pronation/supination (FIGS. 1 and 2) at mid-stance, a second anatomical location to control unwarranted medial or lateral rolling of the foot, specifically protecting at least the Anterior Talofibular Ligament, the Calcaneofibular Ligament, the Posterior Talofibular Ligament, and the Posterior Tibiofibular Ligament. Each strap then extends to bi-laterally grasp the heel, then each flow upward to securely attach to their respective medial/lateral upper aspects of the brace.

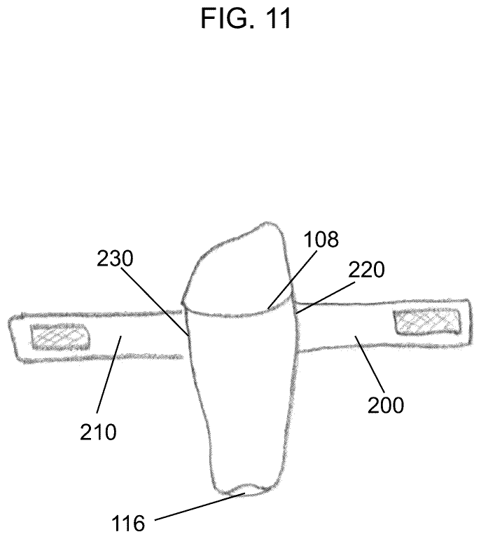

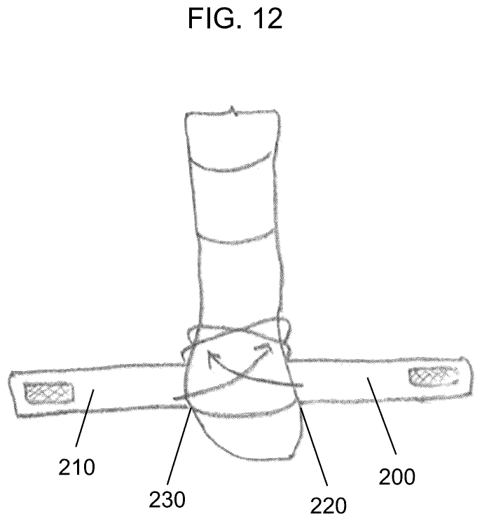

The countervailing force straps can be as shown in FIGS. 4-14 and 24. The countervailing force straps can include a medial countervailing force strap 200 and a lateral countervailing force strap 210. The medial countervailing force strap 200 can be operable to wrap around the foot of the wearer. For example, the medial countervailing force strap 200 can be operable to extend from the medial side near the first aperture (first free edge 104), underneath the foot of the wearer to a lateral side of the body 102, and over the foot to the medial side of the body 102 near the second aperture (second free edge 108). The lateral countervailing force strap 210 can be operable to wrap around the foot of the wearer. The lateral countervailing force strap 210 can be operable to extend from the lateral side near the first aperture (first free edge 104), underneath the foot of the wearer to the medial side of the body 102, and over the foot to the lateral side near the second aperture (second free edge 108).

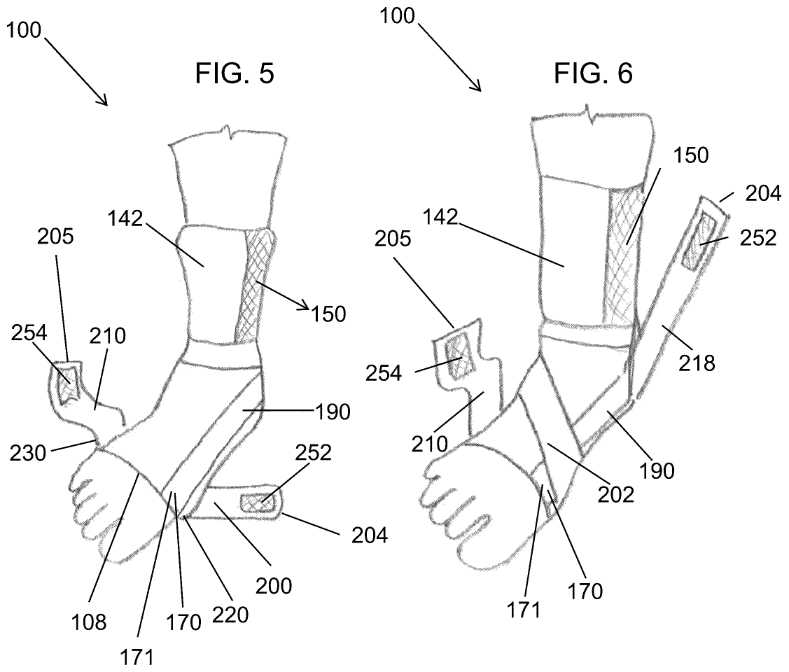

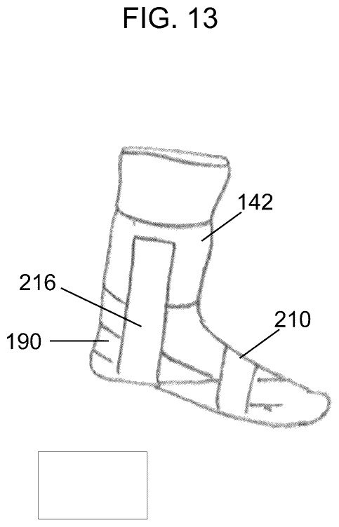

The medial countervailing force strap 200 can be operable to couple to the body 102 near a medial side of the first free edge 104 (e.g., first aperture) and near a medial side of the second free edge 108 (e.g., second aperture). For example, as illustrated in FIGS. 5-6, the medial countervailing force strap 200 can be coupled to a medial side of the foot of the wearer near the second free edge 108 (e.g., near the toes of the wearer) on one end. The medial countervailing force strap 200 can then extend over the foot of the wearer to a lateral side of the body 102 (e.g., opposite side of the foot from the first end of the medial countervailing force strap 200). The medial countervailing force strap 200 can then extend underneath the foot of the wearer (e.g., near the arch of the foot). The medial countervailing force strap 200 can then extend up from the medial side of the body 102 and couple to body 102 near the medial side of the first free edge 104 above the ankle of the wearer. Similarly, the lateral countervailing force strap 210 can be operable to couple to the body 102 near a lateral side of the first free edge 104 (e.g., first aperture) and near a lateral side of the second free edge 108 (e.g., second aperture). For example, as illustrated in FIG. 13, the lateral countervailing force strap 210 can be coupled to a lateral side of the foot of the wearer near the second free edge 108 (e.g., near the toes of the wearer) on one end. The lateral countervailing force strap 210 can then extend over the foot of the wearer to a medial side of the body 102 (e.g., opposite side of the foot from the first end of the lateral countervailing force strap 210). The lateral countervailing force strap 210 can then extend underneath the foot of the wearer (e.g., near the arch of the foot). The lateral countervailing force strap 210 can then extend up from the lateral side of the body 102 and couple to body 102 near the lateral side of the first free edge 104 above the ankle of the wearer.

In some aspects, one end of the medial countervailing force strap 200 and/or the lateral countervailing force strap 210 can be integral to the body 102.

Referring to FIGS. 4-14 and 24, the brace 100 can include a medial countervailing force strap 200 anchored at a medial countervailing force strap anchor location 220 and a lateral countervailing force strap 210 anchored at a lateral countervailing force strap anchor location 230, respectively, at the lateral and medial edge of the second integral sleeve 114 and adjacent the second free edge 108 of the second integral sleeve 114. It is understood that the medial countervailing force strap 200 and lateral countervailing force strap 210 may be attached together to form a single elongated strap anchored at the medial countervailing force strap anchor location 220 and the lateral countervailing force strap anchor location 230 or any location therebetween so as to form a single strap with the medial countervailing force strap 200 and lateral countervailing force strap 210 straps or separate straps extending therefrom. The medial countervailing force strap end 204 of medial countervailing force strap 200 comprises attachment means 252 the medial countervailing force strap end 204 to a first complementary means of attachment on the medial side of brace 100 at a position on the medial side of the brace about the ankle joint. Further, the lateral countervailing force strap end 205 of lateral countervailing force strap 210 comprises attachment means 254 the lateral countervailing force strap end 205 to a second complementary means of attachment on the brace at a position on the medial side of the brace about the ankle joint. In some aspects, the first and second means for attachment on the medial and lateral sides of the brace, respectively, can be a first means of attachment 150 on the medial side of the circumferential strap 142 and a second means of attachment 152 on the lateral side of the circumferential strap 142. As explained above, the first means of attachment 150 is complementary to attachment means 252 at the medial countervailing force strap end 204 of medial countervailing force strap 200, and the second means of attachment 152 is complementary to attachment means 254 at lateral countervailing force strap end 205 of the lateral countervailing force strap 210.

Some aspects of this invention may utilize four or more straps to provide the countervailing forces necessary to prevent excessive supination or pronation of the foot and ankle joint and/or to provide compressive forces on the ankle joint. In some aspects, the medial countervailing force strap 200 and lateral countervailing force strap 210 countervailing force straps are elastic straps. In other aspects, the medial countervailing force strap 200 and lateral countervailing force strap 210 countervailing force straps are not elastic. The medial countervailing force strap 200 and lateral countervailing force strap 210 countervailing force straps can be made from patterns 200′ and 210′, respectively, as illustrated in FIG. 21.

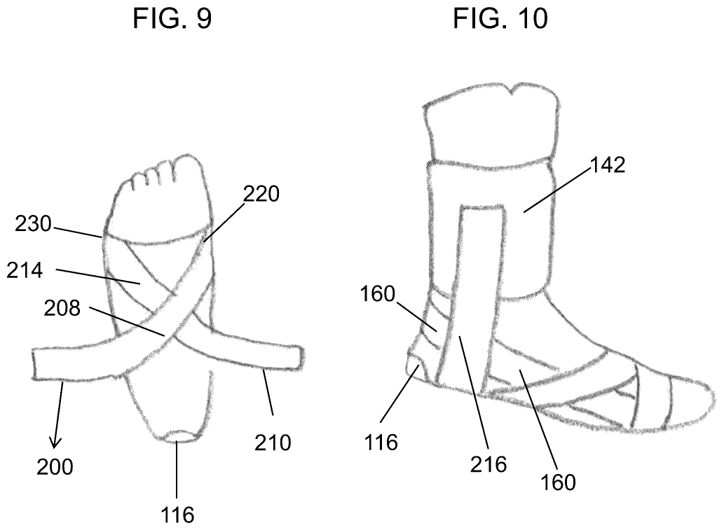

As such, a first extension 202 of medial countervailing force strap 200 extends from medial countervailing force strap anchor location 220 to the medial side of the foot, across the dorsal aspect of the foot and rearwardly towards the superior aspect of the heel (FIG. 6). A second extension 208 of the medial countervailing force strap 200 extends underneath the arch as best shown in FIG. 9. A third extension 218 of the remaining portion of the medial countervailing force strap 200 extends upwardly along the medial side of the ankle joint region as best shown in FIG. 6. The attachment means 252 at medial countervailing force strap end 204 of medial countervailing force strap 200 is attached to a first complementary means of attachment 150 on the brace 100 at a position on the medial side of the brace about the ankle joint. The attachment means can be as described above. In some aspects, the complementary attachment means are complementary hook and loop attachment means. Accordingly, the right foot being shown, the medial countervailing force strap 200 is wrapped around the foot of the wearer so as to provide a force to secure the forefoot, heel and ankle regions of the wearer.

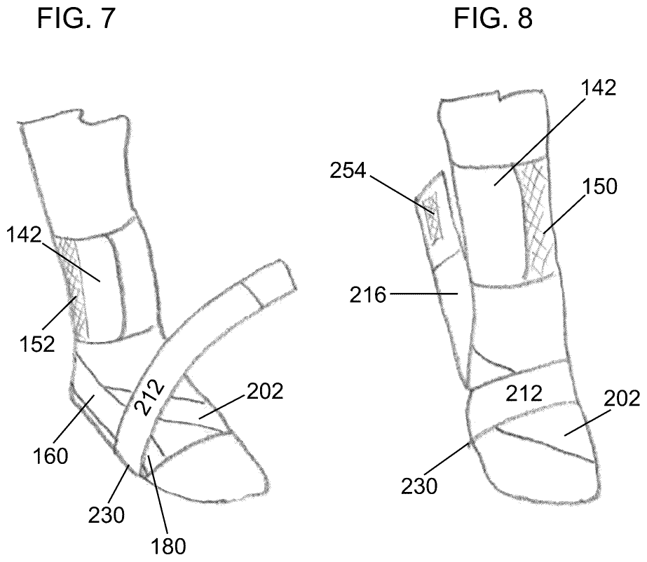

Continuing with the figures as above, a first lateral countervailing force strap extension 212 of lateral countervailing force strap 210 extends from lateral countervailing force strap anchor location 230 above the dorsal aspect of the forefoot and rearwardly towards the medial side of the heel (FIGS. 7, 8 and 12). A second lateral countervailing force strap extension 214 is then wound underneath the arch (FIG. 9) with a third lateral countervailing force strap extension 216 brought upwardly along the lateral side of the heel and ankle joint (FIG. 10). The attachment means 254 at lateral countervailing force strap end 205 of lateral countervailing force strap 210 is attached to a second complementary means of attachment 152 on brace 100 at a position on the medial side of the brace about the ankle joint. The attachment means can be as described above. In some aspects, the complementary attachment means are complementary hook and loop attachment means. Accordingly, the right foot being shown, the lateral countervailing force strap 210 is wrapped just prior to the forefoot of the wearer so as to provide a stabilizing force to the forefoot, heel and ankle regions of the wearer.

In some aspects, the first and second means for attachment on the medial and lateral sides of the brace, respectively, can be a first means of attachment (not shown) on the medial side of the body 102 and a second means of attachment (not shown) on the lateral side of the body 102. As explained above, the first means of attachment is complementary to attachment means 252 at medial countervailing force strap end 204 of medial countervailing force strap 200, and the second means of attachment is complementary to attachment means 254 at lateral countervailing force strap end 205 of lateral countervailing force strap 210.

### (c) Heel Lock and Torque Strap

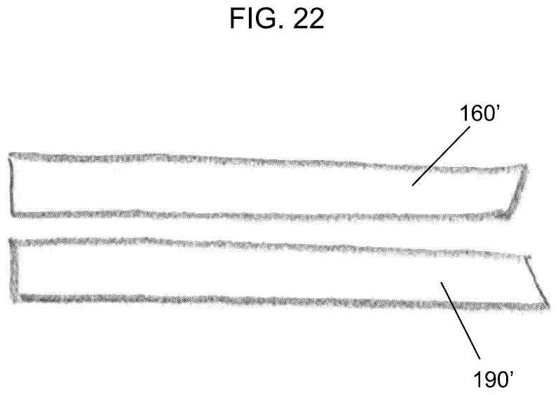

In some aspects, the brace 100 further comprises a lateral heel lock and torque strap 160 and a medial heel lock and torque strap 190. The heel straps function as a stabilizing heel locking mechanism, in a manner similar to an ankle taping procedure. The lateral heel lock and torque strap 160 comprises a first lateral heel lock and torque strap end 180 attached at a first heel lock and torque strap attachment point 181 at the second free edge 108 on the medial side of the body 102 and a second lateral heel lock and torque strap end 182 attached at the rear of the body 102. The lateral heel lock and torque strap 160 extends from the first heel lock and torque strap attachment point 181 along the lateral side of the foot around the heel to the second lateral heel lock and torque strap end 182 at the rear of the body 102. The medial heel lock and torque strap 190 comprises a first heel lock and torque strap end 170 attached at a second heel lock and torque strap attachment point 171 at the second free edge 108 on the medial side of the body 102 and a second medial heel lock and torque strap end 172 attached at the rear of the body 102. The medial heel lock and torque strap 190 extends from the second heel lock and torque strap attachment point 171 along the medial side of the foot to the second lateral heel lock and torque strap end 172 at the rear of the body 102. It is understood that the lateral heel lock and torque strap 160 and medial heel lock and torque strap 190 may be attached together to form a single elongated strap attached at the rear of the body 102 or any location therebetween so as to form a single strap with the medial heel lock and torque strap 190 and lateral heel lock and torque strap 160 or separate straps extending therefrom. The lateral heel lock and torque strap 160 can be made from pattern 160′ and the medial heel lock and torque strap 190 can be made from pattern 190′, as illustrated in FIG. 22.

In some aspects, the lateral heel lock and torque strap 160 and the medial heel lock and torque strap 190 are an integral part of the body 102 and are permanently attached to the body 102 along the length of the lateral heel lock and torque strap 160 and the medial heel lock and torque strap 190. In other aspects, the lateral heel lock and torque strap 160 and the medial heel lock and torque strap 190 are permanently but partially attached (e.g., one side is permanently attached, and the other side is operable to removably couple) at the rear of the body 102. In yet other aspects, lateral heel lock and torque strap 160 and the medial heel lock and torque strap 190 are removably attached to the body 102. When the lateral heel lock and torque strap 160 and the medial heel lock and torque strap 190 are removably or partially attached to the body 102, the lateral heel lock and torque strap 160 and the medial heel lock and torque strap 190 further comprise means of attaching the lateral heel lock and torque strap 160 and the medial heel lock and torque strap 190 to the body 102. Means for removably attaching can be as described above. In some aspects, the lateral heel lock and torque strap 160 and the medial heel lock and torque strap 190 are attached together to form a single heel lock and torque strap 195 comprising a first heel lock and torque strap end 180 attached at a first heel lock and torque strap attachment point 181 and a second heel lock and torque strap end 170 attached at a second lateral heel lock and torque strap attachment point 171.

The medial heel lock and torque strap 160 and the lateral heel lock and torque strap 190 can be operable to secure a subtalar joint of the wearer in subtalar neutral, thereby protecting posterior and anterior tabula fibular and calcaneus fibular ligaments associated with ankle injuries. The medial heel lock and torque strap 160 and the lateral heel lock and torque strap 190 can provide additional control of inversion/eversion movement of a forefoot of the wearer. The additional control provided by the medial heel lock and torque strap 160 and the lateral heel lock and torque strap 190 can prevent excess inversion/eversion, excess pronation/supination, and excess dorsiflexion/plantarflexion.

### (d) Bi-Lateral Heel Straps

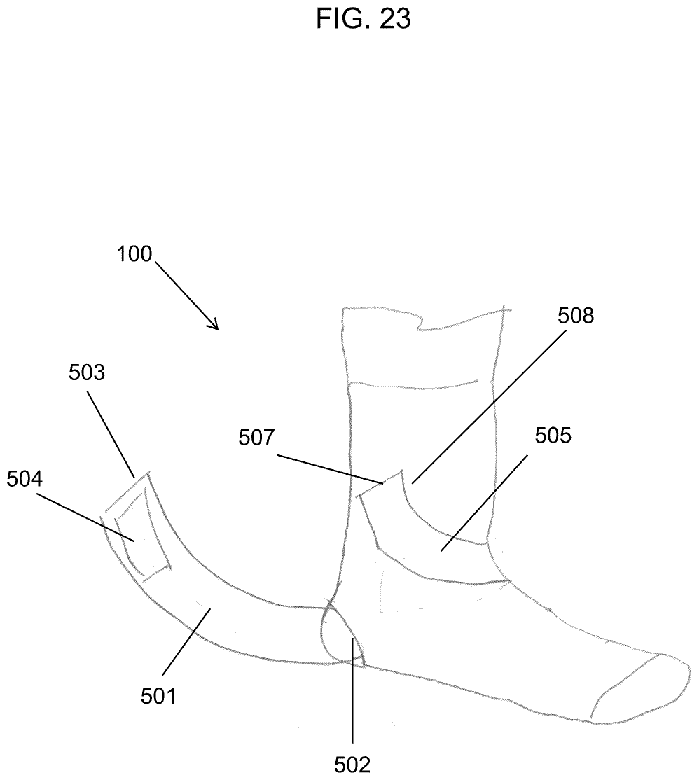

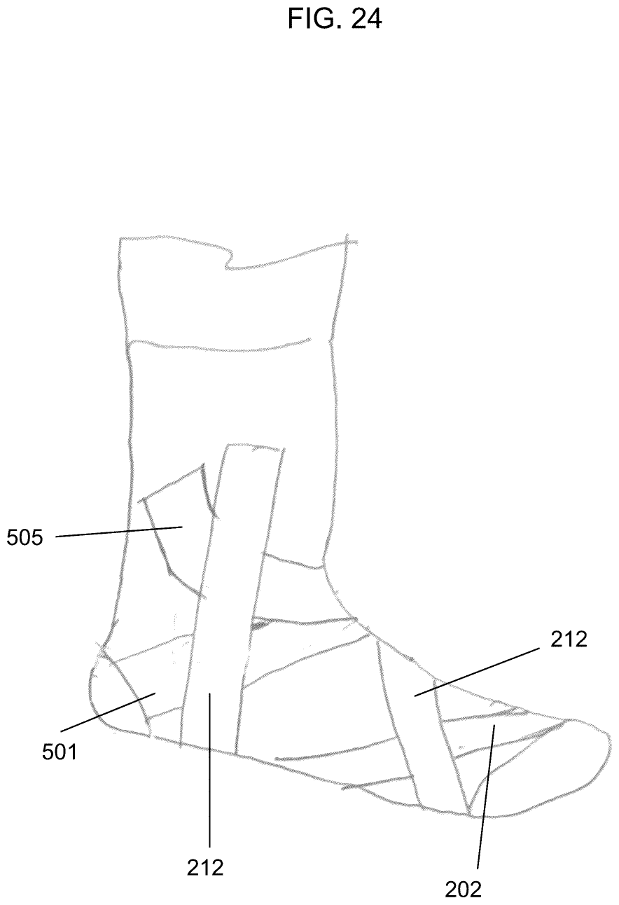

In some aspects, the brace 100 further comprises a first bi-lateral heel strap 501 and a second bi-lateral heel strap 505 as shown in FIGS. 23 and 24. The first bi-lateral heel strap 501 can be operable to couple to the body 102 near a lateral side of a heel of the wearer on one end and at a medial side of the body 102 above the ankle of the wearer at an opposite end. The first bi-lateral heel strap 501 can wrap around a hindfoot and an ankle of the wearer. The second bi-lateral heel strap 505 can be operable to couple to the body 102 near a medial side of the heel of the wearer at one end and at a lateral side of the body above the ankle of the wearer at an opposite end. The second bi-lateral heel strap 505 can wrap around a hindfoot and an ankle of the wearer. The first bi-lateral heel strap 501 and the second bi-lateral heel strap 505 can cross (e.g., one strap under the other) near the hindfoot of the wearer.

The first bi-lateral heel strap 501 comprises a first bi-lateral heel strap first end 502 and a first bi-lateral heel strap second end 503. The first bi-lateral heel strap 501 further comprises a means of attaching 504 the first bi-lateral heel strap second end 503 to the body 102 of the brace 100. The first bi-lateral heel strap first end 502 is attached to the body 102 of the brace 100 at the heel on the lateral side of the body 102 of the brace 100 and the first bi-lateral heel strap second end 503 can attach to the body 102 of the brace 100 at an attachment point (not shown) at a location above the ankle joint complex at the medial side of the body 102 of the brace 100 using the means of attachment. The second bi-lateral heel strap 505 comprises a second bi-lateral heel strap first end 506 and a second bi-lateral heel strap second end 507. The second bi-lateral heel strap 505 further comprises a means of attaching (not shown) the second bi-lateral heel strap second end 507 to the body 102 of the brace 100. The second bi-lateral heel strap first end 506 is attached to the body 102 of the brace 100 at the heel on the medial side of the body 102 of the brace 100 and the second bi-lateral heel strap second end 507 can attach to the body 102 of the brace 100 at an attachment point 508 at a location above the ankle joint complex at the lateral side of the body 102 of the brace 100 using the means of attachment. The attachment means can be as described above. In some aspects, the means of attachment is a loop and hook system, such as Velcro™ fastening material. The first bi-lateral heel strap 501 and the second bi-lateral heel strap 505 are operable to extend from their respective first ends along the midfoot to their respective attachment points on the body 102 of the brace 100. In other examples, the first bi-lateral heel strap 501 and the second bi-lateral heel strap 505 can have fasteners configured to removably couple to corresponding fasteners on medial countervailing force strap 200 and the lateral countervailing force strap 210.

In other aspects, the first bi-lateral heel strap 501 can couple to a medial side of the body 102 near the heel of the wearer. In this aspect, the first bi-lateral heel strap 501 wrap around the back of the heel, around the midfoot, and couple to the body 102 on the medial side above the ankle of the wearer. The second bi-lateral heel strap 505 can couple to a lateral side of the body 102 near the heel of the wearer. In this aspect, the second bi-lateral heel strap 505 can wrap around the back of the heel, around the midfoot, and couple to a lateral side of the body 102 above the ankle of the wearer.

In some aspects, the first bi-lateral heel strap 501 and the second bi-lateral heel strap 505 can be operable to secure the subtalar joint of the wearer in a subtalar neutral position, thereby protecting the posterior and anterior tabula fibular and calcaneus fibular ligaments associated with ankle injuries. In some aspects, the first bi-lateral heel strap 501 and the second bi-lateral heel strap 505 provide additional control of inversion/eversion movement of a forefoot of the wearer. In some aspects, the additional control provided by the first bi-lateral heel strap 501 and the second bi-lateral heel strap 505 prevents excess inversion/eversion, excess pronation/supination, and excess dorsiflexion/plantarflexion. In some aspects, the first bi-lateral heel strap 501 and the second bi-lateral heel strap 505 can stabilize the Achilles tendon of the wearer, thereby enhancing support during movement of the wearer's foot.

### (e) Aspects

In some aspects, a foot and ankle stabilizing brace of the instant disclosure comprises a body, countervailing force straps, and heel lock and torque straps as shown in FIGS. 4-7 and 10. The body 102 provides compressive forces about the ankle and foot of the user. The medial countervailing force strap 200 and lateral countervailing force strap 210 present countervailing forces beginning at the metatarsal heads, along the forefoot and upward along the ankle joint complex. As previously discussed, pronation and supination of the foot respectively present opposed movements at the subtalar joint, hindfoot, and midfoot such that the foot favors the lateral region foot (supination) or the medial region (pronation) of the foot. Medial countervailing force strap 200 and lateral countervailing force strap 210 thus present countervailing forces in these regions to resist movement of the ankle and foot, inclusive of its underlying structures, from excessive pronation or supination. Thus, as the foot is held in a neutral position, the probability of the user landing on an excessively supinated or pronated foot during walking, jumping and/or running is reduced which reduces the probability and/or potential and degree of injury. The use of the medial countervailing force strap 200 and lateral countervailing force strap 210, with or without the compressive fit offered by the body 102 and the compression provided by closure system 120 and circumferential strap 142, enhances the prophylactic advantages of the lateral heel lock and torque strap 160 and the medial heel lock and torque strap 190. The lateral heel lock and torque strap 160 and the medial heel lock and torque strap 190 provide enhanced torque forces to control inversion/eversion movement of the forefoot, stabilizing torque force to the arch, and heel locking forces when attaching the medial countervailing force strap 200 and the lateral countervailing force strap 210 to the brace.

In some aspects, a foot and ankle stabilizing brace of the instant disclosure comprises a body, countervailing force straps, and bi-lateral heel straps as shown in FIG. 24. As discussed above, the body provides compressive forces about the ankle and foot of the user and the countervailing force straps present countervailing forces to resist movement of the ankle and foot, inclusive of its underlying structures, from excessive pronation or supination. The bi-lateral heel straps, in conjunction with the countervailing force straps, unite forces to secure the subtalar joint in subtalar neutral, thereby protecting the posterior and anterior tabula fibular and calcaneus fibular ligaments associated with ankle injuries, including grade I, II & III ankle injuries.

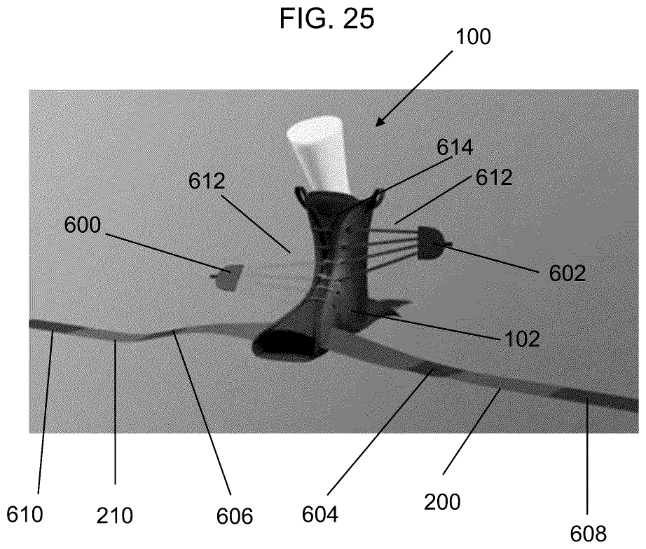

FIG. 25 illustrates an example of a brace 100. The brace 100 can include a tongue 614 and a body 102. The tongue 614 and the body 102 can form a space therebetween for receiving a wearer's foot (e.g., the wearer's foot can be inserted through the first free edge 104 (e.g., first aperture) and the second free edge 108 (e.g., second aperture).

The brace 100 can include laces 612. The laces 612 can be operable to secure the body 102 against the tongue 614 when a wearer's foot is within the brace 100. The laces 612 can be attached to lace fasteners 600, 602. In some examples, the lace fasteners 600, 602 can be operable to attach to the first means of attachment 150 and the second means of attachment 152, respectively. In some examples, the lace fasteners 600, 602 include a hook and loop material operable to removably couple to the first means of attachment 150 and the second means of attachment 152. For example, the first means of attachment 150 and the second means of attachment 152 can include hook and loop material corresponding to the hook and loop material of the lace fasteners 600, 602.

The lace fasteners 600, 602 can provide an initial fit of the brace 100 to the wearer's foot. For example, the lace fasteners 600, 602 can secure the laces 612, and thereby the body 102, around the wearer's foot. In some examples, the lace fasteners 600, 602 and the laces 612 secure the wearer's foot between the tongue 614 and the body 102.

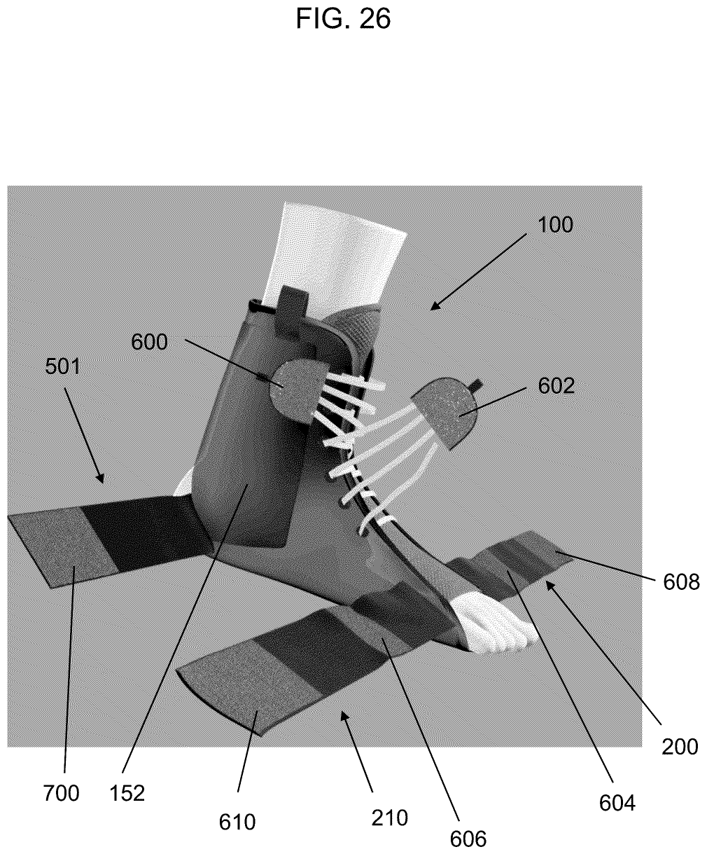

The brace 100 can include a medial countervailing force strap 200 and a lateral countervailing force strap 210, as illustrated in FIGS. 25-26. In some examples, the medial countervailing force strap 200 can include two fasteners configured to fasten to the body 102 at two different points. For example, the medial countervailing force strap 200 can include a fastener configured to fasten to the body 102 at the midfoot and a fastener configured to fasten to the body 102 near the ankle, as will be described further herein. In some examples, the lateral countervailing force strap 210 can include two fasteners configured to fasten to the body 102 at two different points. For example, the lateral countervailing force strap 210 can include a fastener configured to fasten to the body 102 at the midfoot and a fastener configured to fasten to the body 102 near the ankle, as will be described further herein.

In some examples, the lateral countervailing force strap 210 can include a first fastener 606 and a second fastener 610. In some examples, the first fastener 606 and the second fastener 610 can include hook and loop material. In some examples, the second fastener 610 is a double sided fastener (e.g., the second fastener 610 can include hook and loop material on both sides of the second fastener 610).

In some examples, the medial countervailing force strap 200 can include a first fastener 604 and a second fastener 608, as illustrated in FIGS. 25-26. In some examples, the first fastener 604 and the second fastener 608 can include hook and loop material. In some examples, the second fastener 608 is a double sided fastener (e.g., the second fastener 608 can include hook and loop material on both sides of the second fastener 608).

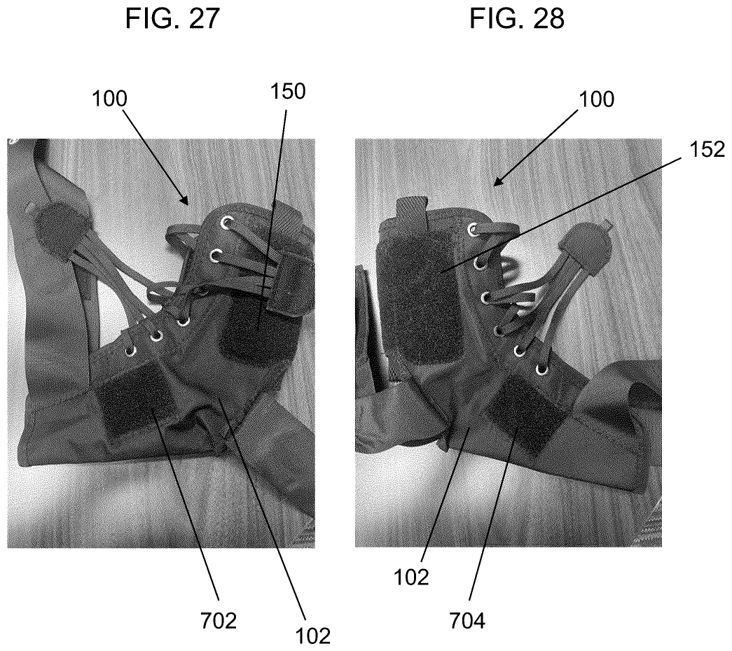

As illustrated in FIG. 27, the body 102 of the brace 100 can include a medial midfoot fastener 702. The medial midfoot fastener 702 can be operable to receive the first fastener 606 of the lateral countervailing force strap 210. For example, the first fastener 606 of the lateral countervailing force strap 210 and the medial midfoot fastener 702 of the body 102 provide a first attachment point between the body 102 and the lateral countervailing force strap 210 as the lateral countervailing force strap 210 is being wrapped around the body 102, and thereby the wearer's foot. By providing this attachment point, the lateral countervailing force strap 210 is secured at the wearer's midfoot, thereby providing additional support to wearer's foot. In this manner, the first fastener 606 and the medial midfoot fastener 702 overcome the limitations of previous brace designs, where straps would slide in use, thereby providing significantly less support to the wearer's foot. Further, the first fastener 606 and the medial midfoot fastener 702 allow the lateral countervailing force strap 210 to stabilize the forefoot and the midfoot of the wearer.

As illustrated in FIG. 28, the body 102 of the brace 100 can include a lateral midfoot fastener 704. The lateral midfoot fastener 704 can be operable to receive the first fastener 604 of the medial countervailing force strap 200. For example, the first fastener 604 of the medial countervailing force strap 200 and the lateral midfoot fastener 704 of the body 102 can provide a first attachment point between the body 102 and the medial countervailing force strap 200 as the medial countervailing force strap 200 is being wrapped around the body 102, and thereby the wearer's foot. By providing this attachment point, the medial countervailing force strap 200 is secured at the wearer's midfoot, thereby providing additional support to the wearer's foot. In this manner, the first fastener 604 and the lateral midfoot fastener 704 overcome the limitations of previous brace designs, where straps would slide in use, thereby providing significantly less support to the wearer's foot. Further, the first fastener 604 and the lateral midfoot fastener 704 allow the medial countervailing force strap 200 to stabilize the forefoot and/or midfoot of the wearer.

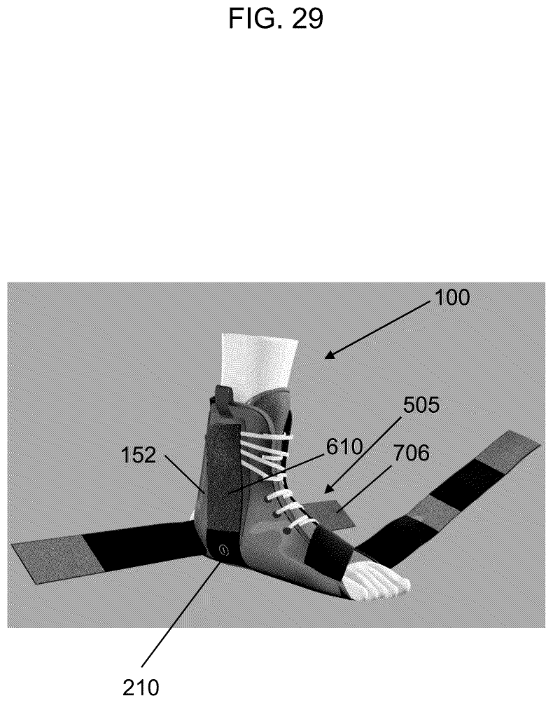

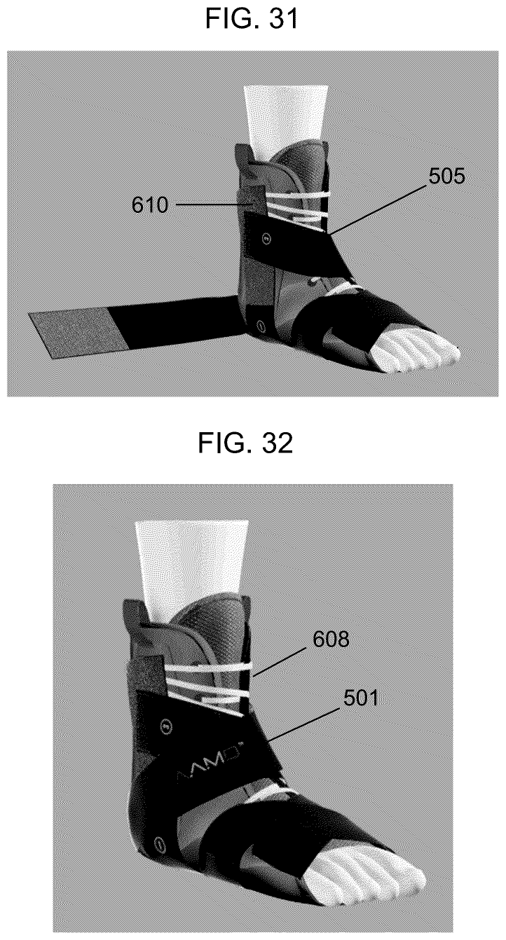

As illustrated in FIG. 29, the lateral countervailing force strap 210 extends over the wearer's forefoot and secures to the medial midfoot fastener 702. The lateral countervailing force strap 210 then extends under the wearer's arch and up to the second means of attachment 152. The second fastener 610 of the lateral countervailing force strap 210 can removably couple to the second means of attachment 152. In this manner, the lateral countervailing force strap 210 is secured around the wearer's foot and ankle. As illustrated in FIG. 29, the second fastener 610 of the lateral countervailing force strap 210 can be double sided, such that the second fastener 610 can removably couple to a heel strap fastener 706 of the second bi-lateral heel strap 505. FIG. 31 illustrates the second bi-lateral heel strap 505 removably coupled to the second fastener 610 of the lateral countervailing force strap 210.

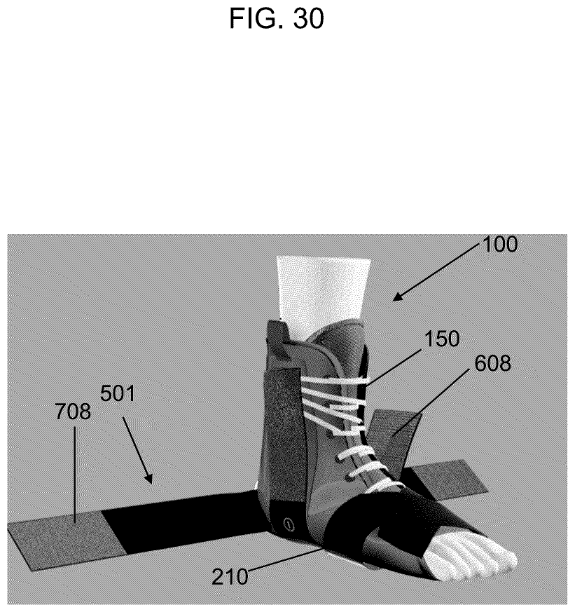

As illustrated in FIG. 30, the medial countervailing force strap 200 extends over the wearer's forefoot and secures to the lateral midfoot fastener 704. The medial countervailing force strap then extends under the wearer's arch and up to the first means of attachment 150. The second fastener 608 of the medial countervailing force strap 200 can removably couple to the first means of attachment 150. In this manner, the medial countervailing force strap 200 is secured around the wearer's foot and ankle. As illustrated in FIG. 30, the second fastener 608 of the medial countervailing force strap 200 can be double sided, such that the second fastener 608 can removably couple to a heel strap fastener 708 of the first bi-lateral heel strap 501. FIG. 32 illustrates the first bi-lateral heel strap 501 removably coupled to the second fastener 608 of the medial countervailing force strap 200.

FIG. 32 illustrates the brace 100 secured to a wearer's foot. The brace 100 can anatomically address conditions clinically credited with forefoot, arch, and ankle conditions. The brace 100 can prophylactically, post injury, and post-surgery, protect the ankle complex from heel strike through toe-lift-off motion. The brace 100 fits bi-laterally to simultaneously stabilize the forefoot, arch, and ankle complex of the wearer.

The strapping mechanisms of the brace 100 provide significant control and stabilization of the wearer's foot. For example, the strapping mechanisms (e.g., medial countervailing force strap 200, lateral countervailing force strap 210, first bi-lateral heel strap 501, and second bi-lateral heel strap 505) control the forefoot from inversion/eversion motion (e.g., first midfoot attachment of the countervailing force straps), reduce pronation/supination of the arch (e.g., extension of countervailing force straps underneath the arch of the wearer), and protect anterior/posterior talar-fibular and calcaneofibular ligament (e.g., connection of countervailing force straps near the ankle and/or bi-lateral heal straps).

In some examples, the fasteners described herein can include medical grade nylon black hook and loop. The body 102 can include terylene oxford fabric. The tongue 614 can include about 12% elastic spandex and about 88% terylene.

### II. Methods

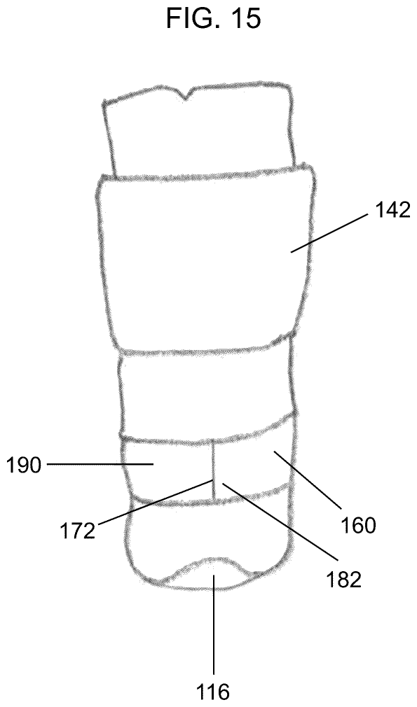

Further provided herein is a method of preventing the excessive supination or pronation of a user's foot and ankle joint by using the stabilizing brace 100. The method comprises having a wearer place their foot into the brace 100 first by placing their foot into the first opening 106 at the first free edge 104 and extending the foot through the second opening 110 of the second free edge 108. The wearer then wraps the medial countervailing force strap 200 around the foot by extending the medial strap through a first extension 202 of the medial countervailing force strap 200 from the medial countervailing force strap anchor location 220 to the medial side of the foot, across the dorsal aspect of the forefoot and rearwardly towards the lateral side of the heel as best depicted in FIG. 15. The medial countervailing force strap 200 is further wrapped by extending through a second extension 208 underneath the arch and through a third extension 218 of the remaining portion of the medial countervailing force strap 200 upwardly along the medial side of the ankle joint region. The wearer then attaches medial countervailing force strap end 204 of medial countervailing force strap 200 to the first complementary means of attachment on the brace at a location on the medial side of the brace above the ankle joint.

The wearer then wraps the lateral countervailing force strap 210 around the foot by extending the medial strap through a first extension 212 of lateral countervailing force strap 210 from anchor location 230 to the medial side of the foot, across the dorsal aspect of the forefoot and rearwardly towards the medial side of the heel. The lateral countervailing force strap 210 is further wrapped by extending through a second extension 214 underneath the arch and through a third extension 216 of the remaining portion of the lateral countervailing force strap 210 upwardly along the lateral side of the ankle joint region. The wearer then attaches lateral countervailing force strap end 205 of lateral countervailing force strap 210 to the second complementary means of attachment on the brace at a location on the lateral side of the brace above the ankle joint.

In some embodiments of the method, when the brace further comprises the closure system 120, the wearer also manipulates the flaps 124, 126 of the notch 122 to adjust the size of the first opening 106 of the first free edge 104 to tighten the fit of the first integral sleeve 112 or to provide additional compressive forces about the ankle joint region.

In some aspects, when the brace further comprises the circumferential strap 142, the method further comprises securing the strap around the body by fastening the left edge 136 to the right edge 137. In one aspect, when the first complementary means of attachment on the brace 100 is at a position on the medial side of the circumferential strap 142, and the second complementary means of attachment on the brace 100 is at a position on the lateral side of the circumferential strap 142, the method further comprises attaching the medial countervailing force strap end 204 of the medial countervailing force strap 200 to the first complementary means of attachment 150 on the circumferential strap 142 and attaching the lateral countervailing force strap end 205 of the lateral countervailing force strap 210 to the second complementary means of attachment 152 on the circumferential strap.

In some aspects, when the brace further comprises the lateral heel lock and torque strap 160 and the medial heel lock and torque strap 190 and the lateral heel lock and torque strap 160 and medial heel lock and torque strap 190 are permanently but partially attached at the rear of the body 102 or removably attached to the body 102, the method further comprises attaching the first lateral heel lock and torque strap end 180 of the lateral heel lock and torque strap 160 to the first attachment point at the second free edge 108 on the lateral side of the body 102, and attaching the first medial heel lock and torque strap end 170 of the medial heel lock and torque strap 190 to the second attachment point at the second free edge 108 on the medial side of the body 102.

In some aspects, when the brace further comprises the first bi-lateral heel strap 501 and the second bi-lateral heel strap 505 of the instant disclosure, the respective first ends of the straps are attached at the rear of the body 102 and the method comprises extending the first bi-lateral heel strap 501 over the front of the ankle and attaching the first bi-lateral heel strap second end 503 to the body 102 of the brace 100 at a location above the ankle joint complex at the medial side of the body 102 of the brace 100 and extending the second bi-lateral heel strap 505 over the front of the ankle and attaching the second bi-lateral heel strap second end 507 to the body 102 of the brace 100 at a location above the ankle joint complex at the lateral side of the body 102 of the brace 100. Although a now preferred embodiment of this invention has been described herein, it is understood that such a description is not limited except as set forth in the claims and allowable, functional equivalents thereof.

In another aspect, a method for precenting excess supination and/or pronation of an ankle is provided herein. At a first step, the method can include providing the foot and ankle stabilizing brace described herein. At a second step, the method can include extending the foot through the first aperture of the foot and ankle stabilizing brace. At a third step, the method can include extending the foot through the second aperture.

At a fourth step, the method can include wrapping the medial countervailing force strap around the foot by extending the medial countervailing force strap from the medial side near the second aperture to a lateral side of the body, underneath an arch of the wearer to the medial side of the body, and up the medial side of the body near the first aperture. At a fifth step, the method can include wrapping the lateral countervailing force strap around the foot by extending the lateral countervailing force strap from the lateral side near the second aperture to a medial side of the body, underneath the arch to the lateral side of the body, and up the lateral side of the body near first aperture. In some examples, the fourth step and the fifth step can be completed in reverse order (e.g., the lateral countervailing force strap is secured first and then the medial countervailing force strap is secured). In some examples, the medial countervailing force strap can include two fasteners, where one fastener attaches to a corresponding fastener on the midfoot of the body and one fastener attaches near the ankle of the wearer. In some examples, the lateral countervailing force strap can include two fasteners, where one fastener attaches to a corresponding fastener on the midfoot of the body and one fastener attaches near the ankle of the wearer.

At a sixth step, the method can include wrapping the first bi-lateral heel strap around the wearer by extending the first bi-lateral heel strap from the lateral side of the heel, around the ankle, around the midfoot, and around the ankle to the medial side of the body above the ankle. At a seventh step, the method can include wrapping the second bi-lateral heel strap around the wearer by extending the second bi-lateral heel strap from the medial side of the heel, around the ankle, around the midfoot, and around the ankle to the lateral side of the body above the ankle.

In some aspects, prior to securing the lateral and countervailing force straps, the method can include tightening the brace using the laces and lace fasteners described herein.

In some aspects, the method can further include crossing the first bi-lateral heel strap and the second bi-lateral heel strap near the midfoot of the wearer. The method can secure a subtalar joint of the wearer in subtalar neutral, via the first bi-lateral heel strap and the second bi-lateral heel strap, thereby protecting posterior and anterior tabula fibular and calcaneus fibular ligaments associated with ankle injuries. The method can provide additional control of inversion/eversion movement of a forefoot of the wearer, via the first bi-lateral heel strap and the second bi-lateral heel strap. The additional control can prevent excess inversion/eversion, excess pronation/supination, and excess dorsiflexion/plantarflexion.

The method can provide countervailing forces along the foot and the ankle to resist excessive supination and pronation thereof, via the medial countervailing force strap and the lateral countervailing force strap.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a,” “an,” “the” and “said” are intended to mean that there are one or more of the elements. The terms “comprising,” “including” and “having” are intended to be inclusive and mean that there may be additional elements other than the listed elements.

As various changes could be made in the above-described cells and methods without departing from the scope of the invention, it is intended that all matter contained in the above description and in the examples given below, shall be interpreted as illustrative and not in a limiting sense.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

What is claimed is:

1. A foot and ankle stabilizing brace comprising:
a body comprising:
a first aperture operable to receive a foot of a wearer, the first aperture above an ankle of the wearer in use; and
a second aperture operable to receive the foot of the wearer, the second aperture at an opposite end of the body from the first aperture;
a medial countervailing force strap operable to couple to the body near a medial side of the first aperture and near a medial side of the second aperture;
a lateral countervailing force strap operable to couple to the body near a lateral side of the first aperture and near a lateral side of the second aperture;
a first bi-lateral heel strap operable to couple to the body near a lateral side of a heel of the wearer and at a medial side of the body above the ankle of the wearer; and
a second bi-lateral heel strap operable to couple to the body near a medial side of the heel and at a lateral side of the body above the ankle;
wherein the first bi-lateral heel strap is operable to wrap around a hindfoot and the ankle of the wearer; and
wherein the second bi-lateral heel strap is operable to wrap around the hindfoot and the ankle.

2. The foot and ankle stabilizing brace of claim 1, wherein the first bi-lateral heel strap and the second bi-lateral heel strap are operable to secure a subtalar joint of the wearer in subtalar neutral, thereby protecting posterior and anterior tabula fibular and calcaneus fibular ligaments associated with ankle injuries.

3. The foot and ankle stabilizing brace of claim 1, wherein the medial countervailing force strap is operable to wrap around the foot of the wearer.

4. The foot and ankle stabilizing brace of claim 3, wherein the medial countervailing force strap extends from the medial side near the second aperture, over the foot to a lateral side of the body, and under the foot to the medial side near the first aperture.

5. The foot and ankle stabilizing brace of claim 4, wherein the medial countervailing force strap includes a fastener operable to couple to a corresponding fastener of the body at the lateral side of the body.

6. The foot and ankle stabilizing brace of claim 1, wherein the lateral countervailing force strap is operable wrap to around the foot of the wearer.

7. The foot and ankle stabilizing brace of claim 6, wherein the lateral countervailing force strap extends from the lateral side near the second aperture, over the foot to a medial side of the body, and under the foot to the lateral side near the first aperture.

8. The foot and ankle stabilizing brace of claim 7, wherein the lateral countervailing force strap includes a fastener operable to couple to a corresponding fastener of the body at the medial side of the body.

9. The foot and ankle stabilizing brace of claim 7, further comprising a tongue and a set of laces attached to the body, the set of laces operable to secure the body and the tongue around the foot of the wearer.

10. The foot and ankle stabilizing brace of claim 1, wherein the medial countervailing force strap and the lateral countervailing force strap are operable to provide countervailing forces along the foot and the ankle to resist excessive supination and pronation thereof.

11. The foot and ankle stabilizing brace of claim 10, wherein the first bi-lateral heel strap and the second bi-lateral heel strap provide additional control of inversion/eversion movement of a forefoot of the wearer.

12. The foot and ankle stabilizing brace of claim 11, wherein the additional control provided by the first bi-lateral heel strap and second bi-lateral heel strap prevents excess inversion/eversion, excess pronation/supination, and excess dorsiflexion/plantarflexion.

13. The foot and ankle stabilizing brace of claim 1, wherein the first bi-lateral heel strap crosses the second bi-lateral heel strap near the hindfoot.

14. A method for preventing excessive supination and/or pronation of an ankle, the method comprising:
providing the foot and ankle stabilizing brace of claim 1;
extending the foot through the first aperture;
extending the foot through the second aperture;
wrapping the lateral countervailing force strap around the foot by extending the lateral countervailing force strap from the lateral side near the second aperture to a medial side of the body, underneath an arch to the lateral side of the body, and up the lateral side of the body near first aperture;
wrapping the medial countervailing force strap around the foot by extending the medial countervailing force strap from the medial side near the second aperture to a lateral side of the body, underneath an arch of the wearer to the medial side of the body, and up the medial side of the body near the first aperture;
wrapping the first bi-lateral heel strap around the wearer by extending the first bi-lateral heel strap from the lateral side of the heel, around the ankle, around the hindfoot, and around the ankle to the medial side of the body above the ankle; and
wrapping the second bi-lateral heel strap around the wearer by extending the second bi-lateral heel strap from the medial side of the heel, around the ankle, around the hindfoot, and around the ankle to the lateral side of the body above the ankle.

15. The method of claim 14, wherein the first bi-lateral heel strap and the second bi-lateral heel strap secure a subtalar joint of the wearer in subtalar neutral, thereby protecting posterior and anterior tabula fibular and calcaneus fibular ligaments associated with ankle injuries.

16. The method of claim 14, wherein the medial countervailing force strap and the lateral countervailing force strap are operable to provide countervailing forces along the foot and the ankle to resist excessive supination and pronation thereof.

17. The method of claim 14, wherein the first bi-lateral heel strap and the second bi-lateral heel strap provide additional control of inversion/eversion movement of a forefoot of the wearer.

18. The method of claim 17, wherein the additional control provided by the first bi-lateral heel strap and the second bi-lateral heel strap prevents excess inversion/eversion, excess pronation/supination, and excess dorsiflexion/plantarflexion.

19. The method of claim 14, wherein the first bi-lateral heel strap crosses the second bi-lateral heel strap near the hindfoot.

20. The method of claim 14, wherein the foot and ankle stabilizing brace further comprises a tongue and a set of laces attached to the body, the set of laces operable to secure the body and the tongue around the foot of the wearer.
